(19) United States
(12) Patent Application Publication
(10) Pub. No.: US 2019/0008441 A1
(43) Pub. Date: Jan. 10, 2019

(54) DIAGNOSING BRAIN INJURY USING A VIRTUAL REALITY SYSTEM

(71) Applicant: VirtualMind, LLC, Missoula, MT (US)

(72) Inventor: Erik E. Guzik, Missoula, MT (US)

(21) Appl. No.: 15/645,241

(22) Filed: Jul. 10, 2017

(51) Int. Cl. A61B 5/00; A61B 5/16

(52) U.S. Cl. CPC A61B 5/4064; A61B 5/6803; G06F 3/0346; A61B 5/163; A61B 5/162

(57) ABSTRACT

System and methods for diagnosing traumatic brain injury in a patient using a virtual reality system. One example embodiment provides a system comprising a virtual reality system and at least one electronic processor. The virtual reality system is comprised of a headset and at least one hand controller configured to receive response input from the patient and generate a patient test response based at least in part on patient input. The at least one electronic processor is configured to select a test-response profile from a plurality of test-response profiles, transmit tests associated with the test-response profile to the headset, receive the patient test response, determine the correctness and timing of the patient test response, create at least one test-response record, and determine, based on comparison of the at least one test-response record to at least one test-response profile, a diagnosis of brain injury for the patient.

### FIELD

Embodiments described herein relate to diagnosing traumatic brain injury by testing a patient with a virtual reality system.

### SUMMARY

Currently, diagnosis of Traumatic Brain Injury (TBI) is done by subjective interpretation of physical symptoms and self-reporting of symptoms. A combination of the Glasgow Coma Scale (GCS) score along with clinical variables such as pupillary reaction and brain scans are often utilized by clinicians but are estimated to be inaccurate for the majority of patients. Clinically, these tests are still widely used because no other validated tests exist to replace them. In short, an objective, rapid, accurate, and reliable cognitive assessment tool is currently lacking to diagnose patients suspected to have TBI. Further, current cognitive testing for TBI often does not provide specific results that lead to actionable information healthcare practitioners can utilize to determine if and when an individual is capable of safely returning to the workplace, battlefield, or competitive athletic activities following an injury. This lack of specific, actionable information often compounds the initial injury by returning individuals to their respective activities too soon, when satisfactory recovery from TBI has yet not occurred. Finally, the lack of accurate test results measuring brain injury, and healing progress, place severe limitations on any future rehabilitation efforts, as effective cognitive treatment and systematic training depends on the reliable measurement of cognitive functioning and development over time.

Accordingly, embodiments described herein test patients for TBI using an cognitive testing environment and testing protocol deployed using a Virtual Reality (VR) system. The system tests patients suspected of suffering a TBI by transmitting a sequence of tests, each test comprised of test data to be delivered to the patient by a VR headset as stimuli intended to generate a response, for example the Field of View test where stimuli is an object in an image on the display of the VR headset, and capturing patient test response input, for example a button depress on at least one hand controller, movement of a hand controller or a VR headset, tracking of at least one eye, capturing eye reaction data, and combinations of these responses. Embodiments described herein capture cognitive responses, cognitive processing speed, the ability to concentrate on cognitive tasks, and other captured data. The cognitive tests are transmitted by an interactive VR system that provides a controlled environment free from visual and audio distractions. The testing solution transmitted by the VR system is a closed system where tests can be presented and results captured effectively. With these results, the system quickly assesses cognitive fatigue, subtle changes in physical coordination, reaction speed, and other reactionary and cognitive data providing accurate and reliable diagnosis of TBI.

Embodiments described herein also allow testing of individual patients prior to sports, battlefield, work, or other activities to establish a baseline that can be compared to results obtained after an incident that suggests a TBI has occurred. Further, the VR system can be used to aggregate test results from groups of patients drawn from a population of similar patients to obtain normative test results for these populations, which can be used when baseline tests for individual patients not previously tested.

The VR testing environment can be controlled and curated by the testing administrator to minimize distractions and isolate specific cognitive functioning during the testing protocol. Alternatively, embodiments of the VR testing system utilize patient specific test results to select and administer tests that more accurately assess specific patients. For example, a patient displaying delayed pupil reaction to visual stimuli in a particular region of their field of view can be tested more fully using these test results allowing a deviation from pre-selected tests that more accurately diagnoses the patient.

One embodiment provides a system for diagnosing TBI using a virtual reality system. In one embodiment the system includes a VR headset worn by the patient and configured to transmit a plurality of tests to the patient and at least one hand controller configured to generate a patient test response input. The electronic processor is also configured to select at least one test-response profile from a plurality of test-response profiles, where a test-response profile consists of a test and an expected response, and transmit the test of the selected test-response profile to the VR headset. The electronic processor is further configured to receive a patient test response to the test from the at least one hand controller, determine correctness and timing of the patient test response and create at least one patient test-response record comprised of the test transmitted to the VR headset and the patient test response data received from the at least one hand controller. The electronic processor is configured to determine, based on comparison of the at least one patient test-response record to at least one expected test-response profile, a diagnosis of brain injury for the patient; and output the diagnosis of brain injury for the patient to a display.

Another embodiment provides a method for diagnosing TBI using a virtual reality system. The method includes selecting, by an electronic processor, at least one test-response profile from a plurality of test-response profiles, where a test-response profile consists of a test and an expected response, transmitting, by the electronic processor, the test from the selected test-response profile to a VR headset, and receiving, by the electronic processor, a patient test response to the test. The method further includes determining, by the electronic processor, the correctness and timing of the patient test response to the test transmitted to the VR headset, creating at least one patient test-response record from the test transmitted to the VR headset and the patient test response received by the electronic processor, determining, by the electronic processor, based on comparison of the at least one patient test-response record to at least one expected test-response record, a diagnosis of brain injury for the patient, and outputting the diagnosis of brain injury for the patient.

### BRIEF DESCRIPTION OF THE DRAWINGS

FIG. 1 schematically illustrates a system for testing patients for traumatic brain injury using a VR system.

FIG. 2a is a flow chart illustrating a method for capturing test results from patients.

FIG. 2b is a flow chart illustrating a method for associating patient feedback with output sent to the virtual reality headset shown in FIG. 1.

FIG. 2c is a flow chart illustrating a method for determining TBI diagnosis for a patient.

FIG. 3 illustrates an example test deployed to the virtual reality testing system of FIG. 1 wherein a patient attempts to track an image on a display in the virtual reality headset of FIG. 1.

FIG. 4 illustrates another example test implementing a Field of View test deployed to the virtual reality testing system of FIG. 1.

FIG. 5 illustrates an example test integrating headphones into the headset attached to the virtual reality headset of the virtual reality testing system of FIG. 1.

FIG. 6 illustrates yet another test integrating eye tracking into the virtual reality headset of the virtual reality testing system of FIG. 1.

### DETAILED DESCRIPTION

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. Furthermore, some embodiments described herein may include one or more processors configured to perform the described functionality by executing instructions stored in non-transitory, computer-readable medium. Similarly, embodiments described herein may be implemented as non-transitory, computer-readable medium storing instructions executable by one or more processors to perform the described functionality. As used in the present application, “non-transitory computer-readable medium” comprises all computer-readable media but does not consist of a transitory, propagating signal. Accordingly, non-transitory computer-readable medium may include, for example, a hard disk, a CD-ROM, a DVD, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

As noted above, diagnosis of TBI takes place by capturing patient test responses and behaviors when presented with tests (stimuli). For example, the Glasgow Coma Scale is a neurological score which intends to provide a reliable and objective way of recording the conscious state of a person for initial as well as subsequent assessment. The score accumulates results from eye reaction data, verbal, and motor responses to stimuli. The Field of View test measures the functional or useful range of a patient's peripheral vision under cognitive load conditions providing another objective method of assessing TBI. Using these, and other tests, physicians diagnose TBI by subjective interpretation of test results, physical symptoms, and patient self-reporting of symptoms, all of which can be error prone, subjective, or even biased as patients often have a great desire to return to their activities.

When a patient is suffering, or suspected of suffering, a TBI, a health professional determines which test(s) to administer, administers the test(s), and accurately captures responses. The health professional analyzes the results to determine a diagnosis. Conflicting responses, inaccurate recording of responses, and other factors influence the resulting diagnosis. Accordingly, as noted above, embodiments described herein transmit tests to patients using a VR system, free of distractions, and objectively capture accurate test responses that reduce interpretation mistakes and patient bias. For example, a Field of View Test can be given by presenting visual objects on the edges of the headset in the VR environment. The patient is then instructed to depress at least one button or provide input through another device (for example an actuator on a hand controller or eye reaction data captured in the headset), depending on an instruction given to the patient in regards to the test (for example, appearance of an image on the right side of the display requires depress of a button on the right hand controller). In the VR environment, the Field of View test can present a visual object in a sequence of locations, gradually moving further away from the center of the headset display screen to accurately determine patient field of view. The patient is unlikely to be able to guess correctly as to when the visual object will appear and thus the VR system reduces bias.

As discussed in more detail below, a VR system automatically captures each test response and stores each test response with other patient test responses. The accumulation of the test responses can be quickly compared to a plurality of previously recorded patient test-response records to provide overall patient results that diagnose or support diagnosis of TBI, or assess recovery from TBI by comparison of previously recorded test results to test results captured after an injury. Accurate test results compiled from analysis of patient test responses collected automatically through the VR system provide objective information to a health professional for analysis and determination of TBI. Further, as also previously mentioned, each test response obtained can be quickly combined with other test responses to select the next test to transmit to the patient.

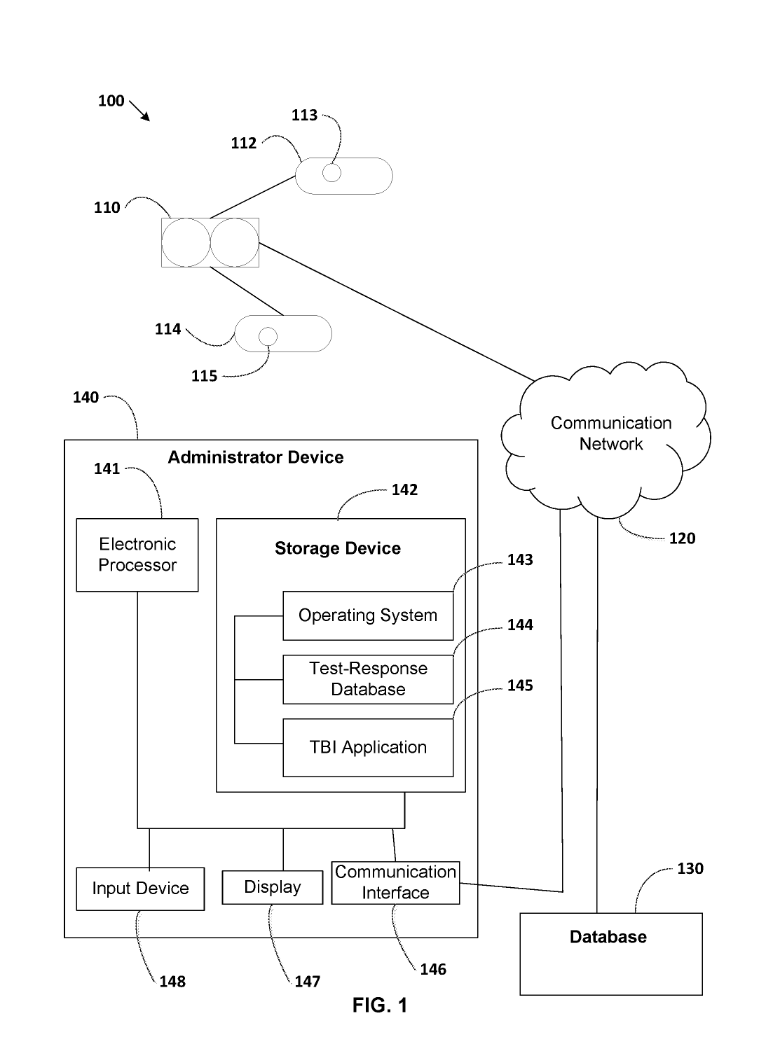

FIG. 1 schematically illustrates a Virtual Reality (VR) system 100 for testing for TBI using a VR environment. As illustrated in FIG. 1, the VR system 100 includes a virtual reality headset 110 worn by a patient, which may be a device with a display screen, a holder for a smart phone or tablet computer, or the like that fits to the patient's head much like googles, and may include or may have the ability to attach earphones, headphones, speakers, or the like to transmit audio to a patient. The virtual reality headset 110 worn by the patient may include or have the ability to integrate an eye tracking system to receive eye reaction data from patient test responses to tests. As also illustrated in FIG. 1, the virtual reality headset 110 communicates with at least one hand controller, for example, a left-hand controller 112, containing a left button 113, and a right-hand controller 114 containing a right button 115. It should be understood communication between the virtual reality headset 110 and both the left-hand controller 112 and the right-hand controller 114 may be wired or wireless. The virtual reality headset 110 communicates over a communication network 120 with a database 130 and an administrator device 140 using one or more communication lines or buses, wireless connections, or a combination thereof.

As shown in FIG. 1, VR system 100 includes the administrator device 140, which may be a laptop or desktop computer, a tablet computer, a smart phone, a smart television, or another type of computing device. As illustrated in FIG. 1, the administrator device 140 includes an electronic processor 141, a storage device 142, a communication interface 146, a output device 147, and an input device 148. The processor 141, the storage device 142, the communication interface 146, the output device 147, and the input device 148 communicate over one or more communication lines or buses, wireless connections, or a combination thereof. It should be understood that, in various configurations, the administrator device 140 may include additional or alternative components than those illustrated in FIG. 1 and may perform additional functions than the functionality described herein. For example, in some embodiments, the administrator device 140 may include multiple processors, storage devices, input devices, output devices, communication interfaces, or combinations thereof.

The processor 141 may include one or more microprocessors, application-specific integrated circuit (ASIC), or other suitable electronic devices. The storage device 142 includes non-transitory, computer readable medium. For example, the storage device 142 may include a hard disk, an optical storage device, a magnetic storage device, ROM (read only memory), RAM (random access memory), register memory, a processor cache, or a combination thereof. The communication interface 146 sends data to devices or networks external to the administrator device 140, receives data from devices or networks external to the administrator device 140, or a combination thereof. For example, the communication interface 146 may include a transceiver for wirelessly communicating over one or more communication networks, for example a wide area network such as the Internet, or a local area network, for example a Bluetooth™ or Wi-Fi network, and combinations or derivatives thereof. Alternatively or in addition, in some embodiments, the communication interface 146 includes a port for receiving a wire or cable, such as an Ethernet cable or a universal serial bus (USB) cable, to facilitate a connection to an external device or network. The output device 147 provides output to a user. For example, the output device 147 may be a light emitting diode (LED), an LED screen, at least one speaker, or the like. The input device 148 receives input from a user. For example, the input device 148 may be a keyboard, keypad, a mouse or trackball, a touchscreen, a microphone, a camera, or the like.

The processor 141 executes instructions stored on the storage device 142 to perform the functionality described herein. The storage device 142 may also store data used with or generated by the execution of instructions by the electronic processor 141. For example, as illustrated in FIG. 1, the storage device 142 may store an operating system 143, a test-response database 144, and a TBI application 145. As noted above, the TBI application 145 executing on the electronic processor 141 selects, or a user selects, or a combination of both the TBI application 145 and the user selects at least one test-response profile from a plurality of test-response profiles stored in the test-response database 144. The at least one expected test-response profile includes at least one test that is sent to the virtual reality headset 110 over the communication network 120 (through the communication interface 146) and the expected test response from the patient. For example, the test-response profile may include a test that presents an image containing a square object on the left side of the headset display, and the expected test response may be depress the button on the right-hand controller.

As noted above and illustrated in FIG. 1, the TBI application 145 receives test responses generated from the left-hand controller 112, the right-hand controller 114, or both, also over the communication network 120, again through communication interface 146. The TBI application 145 associates test responses generated from the left-hand controller 112, the right-hand controller 114, or both, with a specific test transmitted to the virtual reality headset 110, creates a test-response record which is stored in the database 130 through the communication interface 146 over the communication network 120.

It should be understood that the functionality performed by the TBI application 145 is described herein as being performed locally on the administrator device 140. However, this functionality (or portions thereof) may similarly be performed within a distributed environment. For example, in some embodiments, the administrator device 140 may communicate with a server (a cloud service) executing the TBI application 145 or portions thereof. In particular, in one embodiment, the administrator device 140 may access the TBI application 145 executing on a server or a cloud service, which sends tests to the administrator device 140 to be transmitted to virtual reality headset 110, or may transmit the tests to the virtual reality headset 110 across the communication network 120.

In other embodiments, the administrator device 140 may execute the TBI application 145 locally but may access the test-response database 144 located on a server, cloud service, or the database 130 accessed across the communication network 120. Accordingly, it should be understood that the local configuration described in the present application is provided as one example and should not be considered as limiting. In still other embodiments, one or more processors located in the virtual reality headset 110 may execute the TBI application 145, which may access the test-response database 144 located on a server or cloud service, as previously described, or on the administrator device 140. In still other embodiments, one or more processors located in a mobile device, for example a smartphone, attached to the headset may execute the TBI application 145. In this embodiment, the mobile device may access the test-response database 144 across the communication network 120, wherein the communication network 120 may be a wireless communication channel, for example BlueTooth™ or 3G or 4G wireless telephone protocol. In still other embodiments, the test-response database 144 may reside on the mobile device.

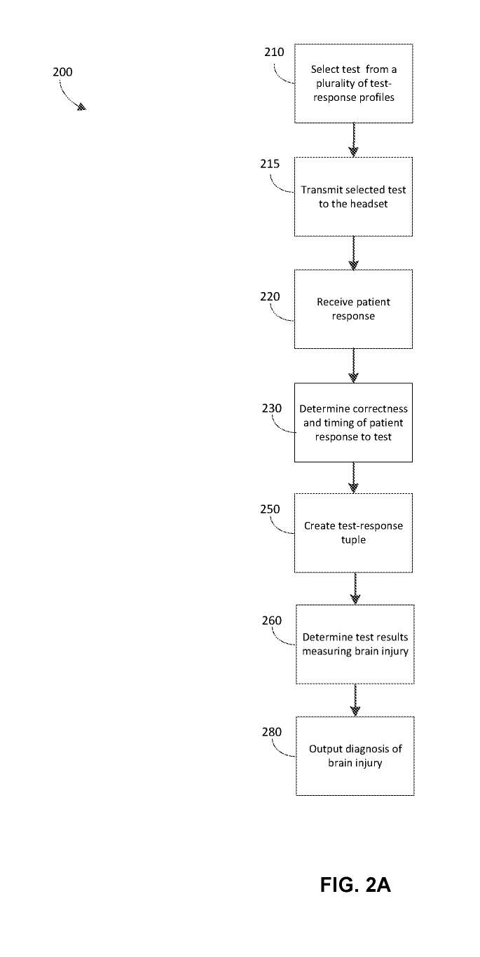

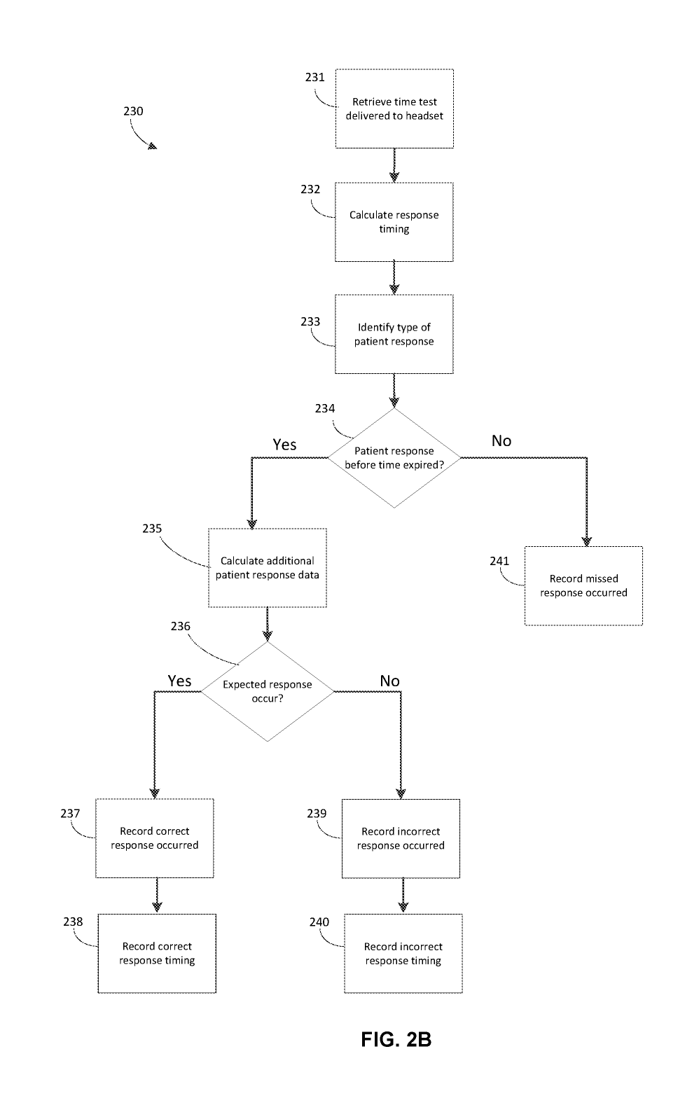

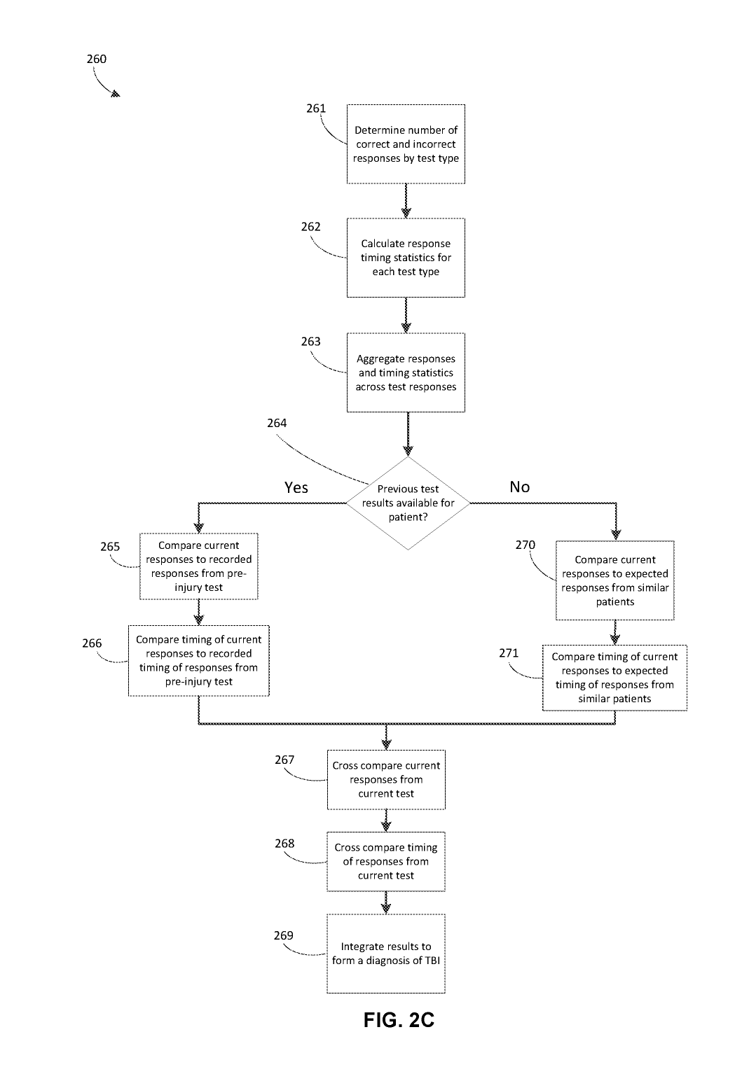

FIG. 2a illustrates a method 200 performed by the TBI application 145 (as executed by the electronic processor 141 included in the administrator device 140, or on a separate computing device, for example a server, or a combination thereof as previously described, or on a mobile device connected to the virtual reality headset 110) to test for brain injury using the VR system 100 according to one embodiment. The method 200 includes selecting at least one test from a plurality of test types (at block 210) from the test-response database 144 as shown, for example, in FIG. 2a. The selection of at least one test from a plurality of tests includes the TBI application 145 executing on electronic processor 141 accessing test-response database 144 and identifying the next test to transmit to the virtual reality headset 110. For example, the TBI application 145 may implement a Field of View (FOV) test which includes a sequence of tests where each test in the sequence consists of a visual image of a shape placed further and further from the patient's center of view. The TBI application 145 implementing the FOV test selects the next test in the sequence to assess the patient's FOV as this aspect of vision can be impacted by a brain injury. In other embodiments, the TBI application 145 executing on electronic processor 141 may receive input from a user through input device 148 to identify the next test to transmit to the virtual reality headset 110. In still other embodiments, the TBI application 145 executing on electronic processor 141 may, by analyzing previous test results, select a set of candidate tests to present to the user on output device 147. In another embodiment, the administrator may review test responses to previous tests and select a test to, for example, retest a patient's test response time to a particular type of test, for example an image displayed briefly in the center of the headset display, or perform additional tests of a patient's inconsistent test responses to audio tests transmitted to the left earphone of the headset. In still other embodiments, the TBI application 145 may identify the next test from a predefined test sequence, for example an audio comprehension test sequence where the patient is sent a sequence of audio tests instructing the patient to depress a specific button on a specific hand controller in response to words that begin with the letter “t”, and the administrator may direct TBI application 145 to select the next test to be a word beginning with a “p” to assess the patient's comprehension versus reaction time.

As shown in FIG. 2a, the method 200 may include the TBI application 145 executing on the electronic processor 141 selecting at least one test from the test-response database 144 (at block 210) and transmitting the at least one selected test to the virtual reality headset 110 (at block 215) as described previously. Transmitting of the test to the virtual reality headset 110 may, for example, include displaying an image on the visual display within the virtual reality headset 110, or playing an audio file on speakers embedded in the virtual reality headset 110, through headphones, or through earphones attached to the virtual reality headset 110, or played in another manner allowing the patient to hear the audio. Further, it should be understood tests transmitted to the virtual reality headset 110 may be in various formats, for example image formats may include jpg, bmp, gif, or other formats, and audio formats may be way, mp3, or the like. Transmitting of the test to the virtual reality headset 110 marks the moment in time when a patient test response occurs, as further described below.

Returning to FIG. 2a, the method 200 includes the TBI application 145 receiving at least one patient test response (at block 220) across the communication network 120, through communication interface 146 on the administrator device 140. The patient test response may be generated from the left-hand controller 112, the right-hand controller 114, at least one accelerometer in either or both hand controllers, or an eye tracking system built into or connected to the virtual reality headset 110, or a combination of test responses from a plurality of these sources. The left-hand controller 112, the right-hand controller 114, or both generate a patient test response from depression, release, or both depressing and releasing the left button 113, the right button 115, or a combination of these actions. In other embodiments, the test response generated may be detection of movement of the left-hand controller 112, the right-hand controller 114, or both using one or more accelerometers. In addition to the patient action with a hand controller button or movement of one or both hand controllers, the time elapsed, determined by comparing the timestamp of transmitting of a test to the virtual reality headset 110 and the timestamp of the patient test response, may be saved in a test-response record, consisting of the test and patient test response data. For example, the patient test response may be comprised of a button depression and the time between the moment in time when test stimuli is delivered to the headset and the moment in time when the patient depresses a button. This pair, in some embodiments, may form the patient test response to a test. It should be understood that a user test response may not occur and such nonoccurrence of a test response within a specified time period after transmitting of the test to the virtual reality headset 110 may be recorded as a missed test response. It should also be understood the time between transmitting of a test and patient action may indicate the patient test response occurred prior to transmitting of the test due to the patient attempting to anticipate the test, or some other reason.

The method 200 includes determining the correctness and timing of a patient test response to a test transmitted to the virtual reality headset 110 (at block 230) as shown in FIG. 2a to create at least one patient test-response record. Further described in FIG. 2b, to determine correctness of the patient test response, the patient test response received from the virtual reality headset 110, the left-hand controller 112, the right-hand controller 114, or a combination of these devices, is compared to the expected test response (at block 230). The timing may be calculated as the elapsed time between transmitting of the test to the virtual reality headset 110 and the time at least one test response is received, or the maximum test response time expires before receiving a test response indicating a missed test response (the patient fails to provide a test response). For example, depression of the right button 115 is received by TBI application 145 after 0.458 seconds has elapsed since transmitting of a visual test to virtual reality headset 110 may be compared to the expected test response in the selected test-response profile. In this example, the visual test is comprised of an image including a white circle on a black background placed on the right side of the display area of the virtual reality headset 110 for which the expected result is left button 113 depress. In this example, the patient test response is <right button, 0.458>. The expected patient test response, in this example, was depress the left button 113 because the test assesses the patient's ability to recognize which side of the display a white circle appears and then to cognitively process the test to depress a button on the opposite hand controller. The patient test-response record created may be <<right-hand, 0.458> opposite hand visual test> (at block 250). In this example the patient test-response record may be aggregated with other patient test-response records by TBI application 145 executing on electronic processor 141 to determine test results measuring brain injury (at block 260).

As shown in FIG. 2a, the method 200 includes the TBI application 145 determining test results to measure brain injury (at block 260). The test-response records created (at block 250) may be stored in the database 130 or on the administrator device 140 in storage device 142, or some combination of both. The TBI application 145 may determine, from at least one statistical result from analysis of test responses, a measurement of brain injury (at block 260) using both the correctness of patient test responses when compared to expected test responses, and the timing of the test responses individually or in aggregate (for example comparing means, variances, and the like). In some embodiments, determining test results measuring brain injury includes comparing current test results to tests completed previously by the same patient, or, in some embodiments, in comparison to previous test results for similar patients (for example, all left hand tests for a patient compared to left hand test results by similar patients), as described in more detail in FIG. 2c.

As shown in FIG. 2a, the method 200 includes the TBI application 145 outputting test results to diagnose brain injury (at block 280). Such output may be presented on display 147 as a probability that a brain injury occurred, or a list specifying the difference between current patient test results and previously recorded test results, or a list showing only test results statistically different from test results from a subset of patient test response records drawn from a population of similar patients, or the like. In other embodiments the diagnosis output may include recommendations, suggestions, or include test results from other types of tests (for example, blood test results).

It should be understood that FIG. 2a illustrates method 200 as one example embodiment of the TBI application 145 and that in some embodiments steps may be combined, for example receiving patient test response (at block 220) and identifying correctness and timing of patient test response (at block 230). In other embodiments, additional steps may be inserted when implementing the method 200 as described in FIG. 2a. For example, test responses may be stored in the database 130 or test-response database 144, or both, and the correctness and timing of each test response determined only after all tests have been transmitted and all test responses stored.

FIG. 2b illustrates further details of identifying the correctness and timing of patient test response as described above with respect to FIG. 2a (see, for example, block 230). The method 230 may begin by retrieving the time a test is transmitted to the virtual reality headset 110 as transmitting indicates the test, for example an image, a sound, or a video, is shown on the display of the virtual reality headset 110, or an audio test is played for the patient (at block 231). The TBI application 145 executing on processor 141 the identifies the correctness and timing of a patient test response to a test transmitted to the virtual reality headset 110. To calculate test response timing, when a test response is received the timing of the test response is captured and the elapsed time from test transmitting to the virtual reality headset 110 and test response received from the virtual reality headset 110, the left-hand controller 112, right-hand controller 114, or some combination of these devices, is calculated (at block 232). In some instances, the patient may fail to respond, in which case the maximum wait time for a test response elapses and the patient test response time may be set to the maximum test response time or another value indicating no test response occurred.

Once test response timing has been calculated (at block 232), electronic processor 141 executing the TBI application 145 identifies the type of patient test response (at block 233). As previously described, the type of test response may be detected movement, for example the direction and speed of movement of the virtual reality headset 110, the left-hand controller 112, the right-hand controller 114, or a combination of these devices, detected by one or more accelerometers in or attached to each device. The patient test response may also be button depress, or button release, or both a depress and release of the left button 113, the right button 115, or a combination of these test responses. Alternatively, or in addition to, the aforementioned test responses, patient test response may be detection of movement of at least one patient eye, pupil, eyelid, or other part of either or both eyes by an eye-tracking system built into, or attached to, the virtual reality headset 110 to capture eye reaction data. In some instances, the patient may fail to respond to the test, in which case the TBI application 145 identifies the type of patient test response (at block 233) as “missed test response” for use in determining brain injury.

As further illustrated in FIG. 2b, if a patient test response occurred before a maximum wait time expires (determined at block 234), additional patient test response data is calculated (at block 235). For example, the time between left button 113 depress and release may also be part of the patient test response, in which case the test response timing is calculated as the time between test transmitting to virtual reality headset 110 and depression of the left button 113, and the time between left button 113 depress and release, and both these times may form additional patient test response data. In another example, the expected patient test response for the test may be to depress the left button 113 and then depress the right button 115. In this example, test response time is calculated as the time between test transmitting to the virtual reality headset 110 and left button 113 depress, and the additional data calculated is the time between the left button 113 depress and the right button 115 depress. In yet another example, the patient test response may occur prior to transmitting of the test to the virtual reality headset 110, which occurs when a patient attempts to guess transmitting of the test to the headset to improve test response times, or for some other reason. In this example, additional data calculated would be the timing between patient test response and test transmitting to virtual reality headset 110, in addition to other data as previously described, or other data, for example multiple patient test responses including a premature test response (prior to transmitting of the test to the virtual reality headset 110) as well as a valid patient test response occurring after the test was transmitted to the virtual reality headset 110).

Returning to FIG. 2b, the patient test response received is compared to the expected patient test response for a specific test (at block 236). If the received patient test response matches the expected patient test response, the TBI application 145 records the received test response as a correct test response (at block 237) and records the test response timing and other data for the correct test response (at block 238). For example, if the received patient test response is the depression and the release of the right button 115, and the expected test response is the depression and the release of the right button 115, the received patient test response matches the expected result. If the time the test was transmitted to the virtual reality headset 110 was 12:11:08.1403 (12^{th} hour, 11^{th} minute, and 8.1403 seconds) and the test response (depress the right button 115) was received at 12:11:08.86 71 (12^{th} hour, 11^{th} minute, and 8.8671 seconds), the test response timing is 0.7268 seconds. If the right button 115 was released at 12:11:08.9783 (12^{th} hour, 11^{th} minute, and 8.9783 seconds), then the additional data calculated measuring the time between depress and release of the right button 115 is 0.1515. The test response is recorded as correct (at block 237), the test response timing (0.7268) and additional data (0.1515) are also recorded (at block 238).

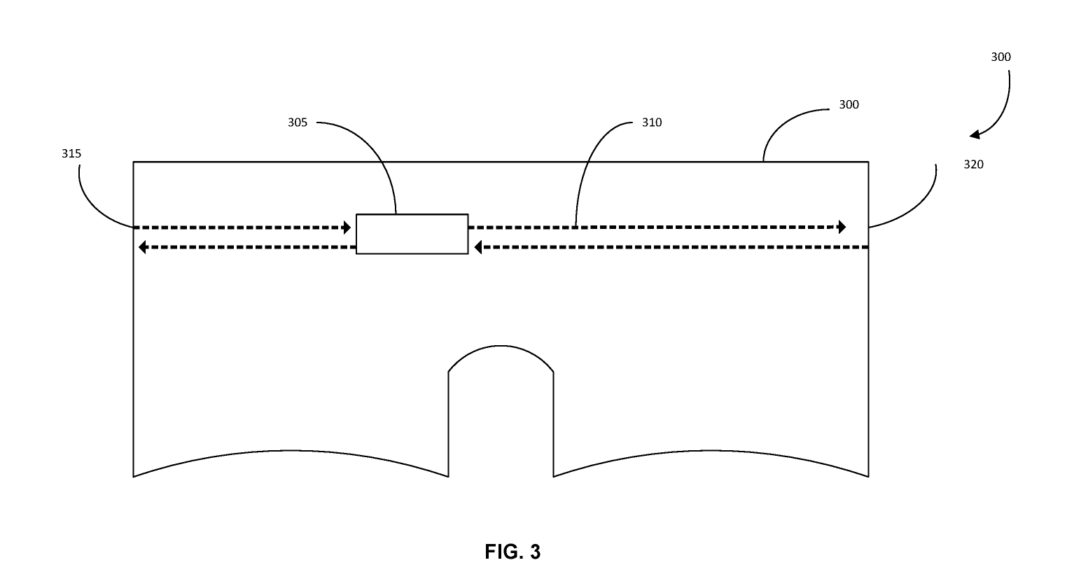

Alternatively, as illustrated in FIG. 2b, if the expected patient test response does not occur (at block 236), the TBI application 145 records the patient test response as an incorrect test response (at block 239) and determines additional data, for example incorrect test response timing, occurrence of multiple test responses, pupil movement, and the like associated with the incorrect test response is recorded (at block 240). Continuing the previous example, if the received patient test response is depress and release left button 113 (on left-hand controller 112), and the expected test response is depress and release the right button 115, the received patient test response does not match the expected test response. If the time the test was transmitted to the virtual reality headset 110 was 12:14:22.4422 (12^{th} hour, 14^{th} minute, and 22.4422 seconds) and the test response (depress button 115) was received at 12:14:23.6458 (12^{th} hour, 11^{th} minute, and 8.8671 seconds), the test response timing is 0.7268 seconds. If the right button 115 was released at 12:11:08.9783 (12^{th} hour, 11^{th} minute, and 8.9783 seconds), then the additional data calculated measuring the time between depress and release of the right button 115 is 0.1515. The test response is recorded as correct (at block 239), and the test response timing (0.7268) and additional data (0.1515) are also recorded (at block 240). It should be recognized that the method 230, as described in FIG. 2b, may occur repeatedly when testing a patient as the testing may include a plurality of tests and the tests may be of different types, as further described in FIGS. 3-6. It should be further understood that the patient test response may include multiple hand controller actions, motions, or combinations of actions and motions, motions or movements of the virtual reality headset 110, or may be multiple test responses detected by an eye tracking system built into or attached to the virtual reality headset 110, or a combination or sequence of these test responses. Testing of a patient may include a plurality of test-response records that may be analyzed (for example accumulated, combined, compared, or otherwise integrated into a statistic or measurement) to detect patient brain functions as manifested in recognition, reaction, processing, and other cognitive actions.

FIG. 2c illustrates further details of a method of diagnosing TBI, in some embodiments, by analyzing patient test results as described above with respect to FIG. 2a (see, for example, block 260). The TBI application 145 captures a plurality of patient test responses and associated test response data illustrated in FIG. 2b and stores this information in a plurality of current patient test-response records as shown in FIG. 2a (at block 250), and the plurality of current patient test responses may be analyzed to determine brain injury. In some embodiments, a plurality of tests of different types may be transmitted to the virtual reality headset 110 and test responses captured. The number of correct, incorrect, premature, and missed test responses may be determined (at block 261) and partitioned by test type or type of expected test response, as specified in an expected patient test response record. For example, two different test types may assess the patient's ability to recognize objects displayed by the virtual reality headset 110 on the left side of the display and respond by depressing the right button 115. One test may display a single object on the left side of the display for a specified period of time, perhaps five seconds, while another test may repeatedly but briefly display an object on the extreme left side of the display, for example one-quarter second every two seconds for six seconds. The expected test response may be a single depression of the right button 115 for the first test and three depressions of the right button 115 for the second test. Determining correct and incorrect observed test responses by test type (at block 261) supports determination of cognitive function when observing stimuli (as a plurality of tests) and then deciding on the appropriate test responses.

Once the number of correct and incorrect test responses have been determined, example embodiment of method 250 illustrated in FIG. 2c may calculate test response timing statistics for each test type (at block 262). Calculation of test response timing statistics may include calculating an average test response time for correct and incorrect test responses, a variance in test response times, differences in average test response times for correct and incorrect test responses, and the like. For example, average timing for correct test responses when observation of an object on one side of the display calls for a reaction on the opposite side hand controller may be lengthy, indicating slower test response when cognitive processing is required by the patient to produce a test response.

The number of correct and incorrect test responses as well as calculation of patient test results timings statistics for each test type may be aggregated by method 260 (at block 263) as illustrated in FIG. 2c. Aggregation of test responses and timing statistics may be pre-set to include a standard group of measures or may be the guided or directed by analysis of test responses. For example, for a baseline test of patient cognitive abilities, a subset of tests may be selected that are aggregated using a preselected approach focused on correctness and timing of test responses without test type analysis or cross comparison of timing results (for example, comparing all left hand responses for consistency of reaction time). Alternatively, aggregation may be driven by variability in observed test responses. For example, when right hand test responses are slower than left hand test responses for reactionary tests (tests not assessing cognitive processing), all tests with expected test responses from right hand controllers may be aggregated. Or, if all tests include cognitive analysis to produce correct results (for example, object displayed on the left, press the right button 115, and vice versa) show higher rates of incorrect test responses, those types of tests may be aggregated. It should be understood that in some cases patient test responses can be aggregated in other ways than described here to produce a set of test results and that these tests results can be compared to previous test results for a patient, or compared to aggregated test results from similar patients, or both. In some embodiments, a set of patient test-response records may be selected from a plurality of patient test-response records for analysis, for example only test-response records based on eye tracking may be selected, or only right button 115 test-response records may be selected.

As illustrated in FIG. 2c, aggregated test responses and timing statistics may be compared to previous test results for a patient, if test results exist for the patient (as tested at block 264). If previous test results exist, current test responses and timing can be compared to patient results from similar tests prior to the occurrence, or suspected occurrence, of a brain injury (at block 265). Test responses from a previously recorded test using the TBI application 145 shown in FIG. 1 may establish a baseline for the patient, meaning, the patient may establish a normative baseline for correctness for specific test types (for example, cognitive tests with expected test response of depress the left button 113) and aggregated test types for example, all tests with expected test response of depress the left button 113). In some instances, for example, a patient may have a higher incorrect test response rate than another patient for left hand controller actions but this may be normal for the particular patient. For example, the percentage of correct test responses to tests calling on a patient to depress any button upon appearance of a circle on the display of the virtual reality headset 110, may be statistically compared using McNemar's test for paired nominal data or z-test for the difference between two proportions.

The TBI application 145 illustrated in FIG. 1 may access baseline tests for patients that can be used to assess brain function following a brain injury using patient specific baseline test results. With patient specific baseline test results available, less false positives may occur in situations where, for example, left hand controller accuracy may be lower than right hand controller accuracy but that result may be normal for a specific patient and thus not an indication of brain injury. Alternatively, if the left hand controller accuracy were to be reduced in a statistically significant amount following a suspected brain injury, that result may indicate a brain injury occurred.

Similar to comparing test response accuracy between previous test responses and at least one current patient test response record, comparing timing results from a current test responses to a pre-injury test responses (at block 265) as illustrated in FIG. 2c may involve statistical tests. For example, the Student's t-test may be used when the variances of both the current test response timing for one or more types of tests and previous test response timing are equal or the Welch's t-test when the variances are not equal. If the current test and previous test are identical in test type and test order, a paired t-test may be used to compare results and determine if differences exist in patient timing test responses to tests. It should be understood alternative or additional statistical tests may be used to compare results, as well as heuristics based on logical relationships, machine learning techniques, and the like may also be used to compare test responses.

As shown in FIG. 2c, comparison of current test responses to previous test responses may include cross comparison of results within current test results (at block 267) to validate that test responses across similar test types and between different test types are consistent. For example, the rate of correct test responses for tests calling on a patient to depress any button upon the appearance a square on the headset display, may be tested for consistency using McNemar's test for paired nominal data or z-test for the difference between two proportions. It should be understood that alternative or additional statistical tests may also be used to assess consistency of test results.

Similarly, the timing of subgroups selected from current test responses may be cross compared to other, separately selected, test responses from current test responses to assess consistency (at block 268), as illustrated in FIG. 2c. As previously discussed, tests used to compare current test responses to previous test responses (at block 265) can be applied to subgroups of tests selected from current test responses. For example, as previously described, the Student's t-test may be used or the Welch's t-test may be used to compare subgroups and determine if differences exist in patient timing test responses to tests. It should be understood alternative or additional statistical tests may be used to compare results, as well as heuristics based on logical relationships, machine learning techniques, and the like may also be used to compare test responses.

Using previously recorded test results for a patient, the TBI application 145 may integrate results of a plurality of test response analyses and comparisons between current test results and previously recorded test results to determine a diagnosis of brain injury (at block 269). Integration of statistical results may include presentation of results of each type of statistical test for user review, may utilize heuristics in the form of rules to determine a diagnosis, or may use artificial intelligence to analyze and integrate both statistical and raw data to form a diagnosis, or may integrate some combination of these approaches to determine a diagnosis. For example, a specific diagnosis may be determined when both a statistically significant difference between previously recorded test responses and current test responses for right hand accuracy across tests that require right-hand test responses and a statistically significant difference between previously recorded and current timing test responses to tests presenting images on the right side of the display in virtual reality headset 110. This diagnosis may result from both statistical results and a heuristic that, for example, determines an injury to a particular part of the brain has occurred when this integration of test results is performed. Alternatively, integration of test results that show a statistically significant change in timing test responses to all tests requiring cognitive processing between display of a test and choosing the correct test response may indicate a specific type of injury regardless of the type of test assessing cognitive responsiveness. It should be understood that test response data may be analyzed using additional or alternative statistical, heuristic, and artificially intelligent methods than those presented here.

Previously recorded test results for a patient may not be available. As shown in FIG. 2c, when no such test results exist (as tested at block 264) the TBI application 145 may compare current test responses to expected test responses from similar patients (at block 270). For example, if the patient is a 16 year old female volleyball player, normalized expected results from female athletes aged 15-16 years of age may be used to compare with current test responses. Alternatively, test responses from a group of patients drawn from a population of patients similar to a current patient, for example previously tested female athletes 16 years of age, may be used to derive expected test responses that can be compared to current test responses. It should be understood that the definition and population of similar patients may be determined in multiple ways and that multiple similar populations may be used for comparison with current results. It should also be understood that previously described comparison and analysis techniques (as described for block 265) may be used to compare current test responses with expected test responses aggregated (for example, average correctness and timing) from groups of patients drawn from a population of similar patients.

Similarly, the timing of current test responses may be compared with timing of test responses from similar patients (at block 271) as shown in FIG. 2c. Similar populations of patients may be defined as previously described and comparison and analysis techniques may be used to compare current test responses with expected test responses from similar patients (as described for block 266). Once comparisons have been made using test responses from similar patients, analysis continues as previously described (blocks 267, 268, and 269) to determine a diagnosis of TBI.

In still other embodiments, the TBI application 145 may compare a sequence of patient test-response records to a sequence of previously recorded patient test-response records from the same patient, or similar patients. In this embodiment the two sequences, current patient test responses and previously recorded test responses can be compared using a pairwise approach and the differences between pairs accumulated and analyzed for brain injury.

Alternatively, the VR system 100 may be used to transmit a limited number of pre-selected tests to the patient over a short time span to assess whether additional testing may be required to more fully determine a diagnosis of brain injury. In this example embodiment, the number of tests transmitted to the patient may be pre-selected and be small in number, for example 5-10 tests given within 5-10 minutes. Integration of the test results in this instance may be compared to previous test results for the same patient, as previously described, or compared to a similar group of patients, also as previously described, or both. In this example embodiment, the results may not be sufficient to diagnose brain injury but may instead be used as a rapid method for assessing the probability a brain injury has occurred and, based on the probability, outputting a recommendation as to the need for additional testing to diagnose brain injury. For example, eight (8) tests may be transmitted to the patient where four (4) tests assess reaction time for of the patient and four (4) tests assess cognitive processing. The reaction time tests may include two tests that present a round object in an image on and require depression of either button on the hand controller while the cognitive tests require depression of the button on the left controller when the object is a circle and depression of the button on the right controller if the object is a square. In this example embodiment, the tests may be transmitted to a headset in the VR system 100 located on the sideline of a sporting event, for example a football game or a soccer match, to quickly provide medical personnel information as to probability a head injury has occurred and the need for additional testing. The additional testing may include a full range of tests, with patient test response feedback used to select tests, add tests, or extend the testing as needed.

Example tests that assess brain injury used in the TBI application 145 are illustrated in FIGS. 3-6. FIG. 3 illustrates an embodiment of a test transmitted to virtual reality headset 110 wherein the VR display 300 presents to the user a sequence of images which together animate movement of an object 305 that moves along a path 310 from a left edge 315 to a right edge 320 of the VR display 300, and then back to the left edge 315 at least once. A patient observes the movement of the object 305 and the expected test response is depression of the left button 113 when the image 305 touches the left edge 315 and depression of the right button 115 when the object 305 touches the right edge 320. This example embodiment of a test on the VR display 300 of the virtual reality headset 110 assesses cognitive ability of a patient by assessing a patient's ability to anticipate an event as well as reaction time based on where the object 305 is when the button depression occurs. It should be recognized other tests may be conducted with this pattern of movement and that additional diagnoses may be supported using the example expected test responses or alternative expected test responses. For example, the patient may be instructed to only depress the right button 115 when the object 305 touches the right side 320, or when the object 305 touches the left side 315, or given other instructions. It should be understood this example test can be configured in different ways, for example changing the size, location, color, and other aspects of the object 305.

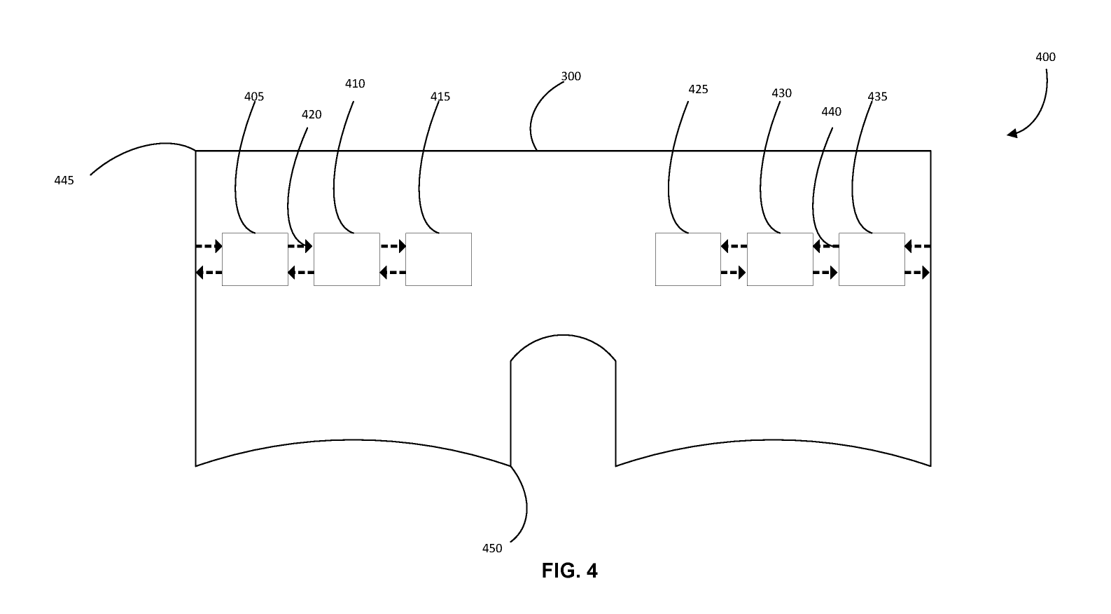

FIG. 4 illustrates another example embodiment of a test that the TBI application 145 may transmit to the virtual reality headset 110 wherein VR display 300 presents to the user a sequence of images which together appear to move objects 405, 410, 415 along a path 420, and, alternatively or simultaneously, move objects 425, 430, and 435 along a path 440. This test can be used, for example, to implement a Field of View test wherein the patient is instructed to depress the right button 115 each time the objects 425, 430, or 435 stops, or becomes visible, along the path 440. The TBI application 145 may transmit variations of the test virtual reality headset 110 quickly and easily. For example, if the patient fails to respond when the object 405 is presented in an image on VR display 300, the TBI application 145 may present the object 410, detect a test response, and may present the object 405 again to test if the patient merely missed responding or there is a consistent Field of View issue wherein the patient cannot see object 405 within an image on the VR display 300 when object 405 appears in certain locations within the image. It should be understood this example test can be configured in different ways, for example changing the size, location, color, and other aspects of the objects 405, 410, 415, 425, 430, and 435. It should also be recognized that the path 420 and path 440 may be altered in location within the image presented on the VR display 300 and follow alternative paths within the image, for example moving the path 420 to the top of the image presented on the VR display 300 or following a path from an upper left corner 445 to a bottom left corner 450 of the image presented on the VR display 300. It should be further understood other tests may be transmitted to the virtual reality headset 110 by the TBI application 145 by, for example, instructing the patient to provide different expected test responses including depression of alternative buttons, release of both buttons, movement of either or both controllers, or a combination of these or other test responses.

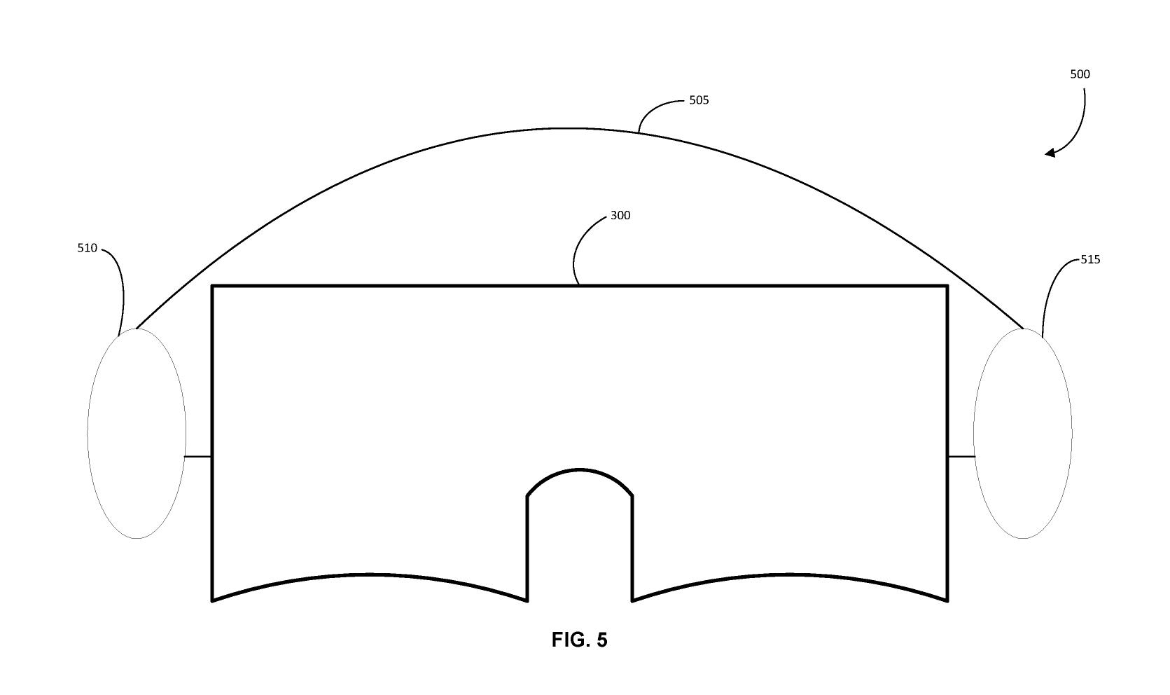

An audio test may also be transmitted to virtual reality headset 110 when headphones 505 are attached to or part of virtual reality headset 110, as shown in example embodiment 500, illustrated in FIG. 5. The headphones 505 include a left speaker 510 and a right speaker 515 that may be used to transmit audio stimuli as a plurality of sounds, for example beeps, words, instructions, and the like, to a patient's ears. For example, each time a sound is transmitted to either the left speaker 510 or the right speaker 515, the patient may be instructed to depress the right button 115. As another example, the patient may be instructed to push the left button 113 when the sound occurs in the left speaker 510 and the right button 115 when the sound occurs in the right speaker 515. Yet other tests may include transmitting a sequence of words to both the left speaker 510 and the right speaker 515 wherein the patient is only to respond if a word in the sequence is a type of fruit or other predetermined category. Still other tests may combine visual and audio components wherein the patient responds only when the visual and the audio occur simultaneously or complement each other in some way (for example the audible word “orange” is output and an image of an orange is display). It should be understood that there exist many variations of audio and visual tests that can be transmitted to virtual reality headset 110 for presentation on VR display 300 and play on the left speaker 510, right speaker 515, or both.

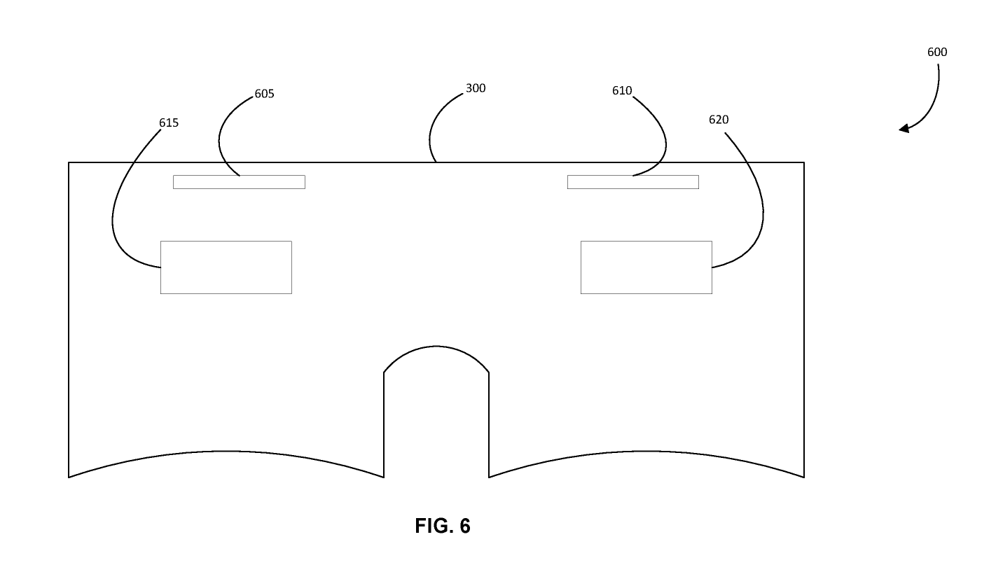

As illustrated in FIG. 6, the virtual reality headset 110 may include eye tracking through a left eye tracker 605 and a right eye tracker 610. Tests using eye tracking may include, for example, presentation of an object 615, an object 620, or both, on the VR display 300. For example, the object 615 may be presented in an image on the VR display 300 and the object 615 moved, through presentation of a series of images sent by TBI application 145 to VR display 300 while a left eye tracker 605 and a right eye tracker 610 monitor eye pupil movement capturing, for example, the ability of the patient to maintain focus on the object 615. If the patient fails to track the object 615, the TBI application 145 may identify this inability to track the object 615 as an incorrect test response. Alternatively, the left eye tracker 605 or the right eye tracker 610 may detect a lag between the movement of the object 615 across multiple images, for example the object 615 appears in a different location and the patient's pupils move to focus on the new location after a timing delay. This delay between the appearance of the object 615 and movement of one or both eye pupils may be recorded as timing, as previously discussed for hand controller button depresses. It should be understood the object 615 is one example object and that objects may be other shapes or sizes, be presented in a variety of colors, and the like. It should also be understood that movement or the location of an object in an image may be occur in a variety of ways, for example appearance in different portions of the image transmitted by TBI application 145 to the VR display 300, or along paths across the VR display 300 through presentation of a sequence of images with the object in different locations.

Thus, embodiments described herein provide methods and systems for testing patients for TBI using a cognitive testing environment and testing protocol deployed using a VR system. The VR system tests patients suspected of suffering a TBI by transmitting a sequence of tests to the patient in the VR system using a headset, which may include a display, headphones, or both, wherein the headset is configured to deliver test stimuli to the patient. The TBI application 145, executing on the electronic processor 141, transmits these tests, receives patient test responses (which may be lack of a test response), analyzes patient test responses, and compares patient test responses to test responses previously recorded from the patient or test responses from similar patients, or both. The TBI application 145 executing on the electronic processor 141 and using a VR system determines a diagnosis of brain injury which is output to the user through an output device.

Various features and advantages of some embodiments are set forth in the following claims.

What is claimed is:

1. A system for diagnosing a brain injury of a patient, the system comprising:
a virtual reality headset worn by the patient and configured to deliver stimuli to the patient, the stimuli associated with a plurality of tests;
at least one hand controller configured to:
receive patient test response input from the patient, the patient test response input generated by the patient at least in part in response to the stimuli; and
generate a patient test response, the patient test response based at least in part on the patient test response input;
an electronic processor configured to:
select at least one test-response profile from a plurality of test-response profiles, a test-response profile consisting of a test and an expected response;
transmit test data associated with the selected test-response profile to the virtual reality headset;
receive the patient test response from the at least one hand controller;
determine correctness and timing of the patient test response to the test transmitted to the virtual reality headset;
create at least one test-response record, the at least one test-response record associated with the test data transmitted to the virtual reality headset and the patient test response;
determine, based on comparison of the at least one test-response record to at least one test-response profile, a diagnosis of brain injury for the patient; and
output the diagnosis of brain injury for the patient.

2. The system of claim 1, wherein the at least one hand controller comprises at least one button for receiving the response input from the patient.

3. The system of claim 1, further comprising an eye tracking system associated with the virtual reality headset and configured to capture eye reaction data from at least one eye of the patient, wherein the patient test response is further based at least in part on the reaction data from at least one eye of the patient.

4. The system of claim 1, further comprising at least one accelerometer associated with the at least one hand controller and configured to detect movement of the at least one hand controller, wherein the patient test response is further based at least in part on the detected movement.

5. The system of claim 1, wherein the plurality of test-response profiles comprises at least one test-response record associated with a test previously transmitted to the patient using the virtual reality headset.

6. The system of claim 1, wherein the expected response is determined, at least in part, using a statistical analysis of test responses received from a group of patients drawn from a population of patients similar to the current patient.

7. The system of claim 1, wherein the test data comprises visual stimuli transmitted to a display of the virtual reality headset.

8. The system of claim 1, wherein the test data comprises audio stimuli transmitted to at least one speaker associated with the virtual reality headset.

9. The system of claim 1, wherein the timing of the patient test response is calculated, at least in part, by measuring the time elapsed between the moment in time the stimuli was transmitted to the patient and the moment in time when the patient test response to the stimuli is detected.

10. The system of claim 1, wherein the electronic processor is further configured to compare data from a plurality of previously recorded patient test-response records to at least one current patient test-response records.

11. The system of claim 1, wherein the electronic processor is further configured to compare a sequence of patient test-response records with a sequence of previously recorded patient test-response records.

12. The system of claim 1, wherein the electronic processor is further configured to compare at least one current patient test-response record with a plurality of test-response records associated with a population of patients similar to the patient being diagnosed.

13. The system of claim 1, wherein the electronic processor is further configured to analyze a subset of patient test-response records selected from a plurality of patient test-response records and compare the results with test results aggregated from previously recorded patient test-response records.

14. A method for diagnosing a patient for brain injury, the method comprising:
selecting, by an electronic processor, at least one test-response profile from a plurality of test-response profiles, where a test-response profile consists of a test and an expected response;
transmit, by the electronic processor, test data from the selected test-response profile to a virtual reality headset;
receiving, by the electronic processor, a patient test response to the test data;
determining, by the electronic processor, correctness and timing of the patient test response to the test data transmitted to the virtual reality headset;
creating at least one patient test-response record, the at least one patient test-response record associated with the test data transmitted to the virtual reality headset and the patient test response received by the electronic processor;
determining, by the electronic processor, based on comparison of the at least one patient test-response record to at least one test-response profile, a diagnosis of brain injury for the patient; and
outputting the diagnosis of brain injury for the patient.

15. The method of claim 14, wherein selecting, by the electronic processor, at least one test-response profile from a plurality of test-response profiles includes analyzing previous test results from the patient to detect when further testing should be done, and, if further testing should be done, selecting at least one test to transmit to the patient.

16. The method of claim 14, wherein receiving the patient test response includes receiving, by the electronic processor, at least one patient test response from the at least one hand controller selected from a group consisting of a button depress, a button release, and movement of the accelerometer of the at least one hand controller.

17. The method of claim 14, wherein determining, by the electronic processor, the correctness and timing of a patient test response to test data transmitted to the headset includes:
retrieving the time the test data was transmitted to the virtual reality headset,
calculating, at least in part, the timing of the patient test response by measuring the time elapsed between the moment the stimuli was delivered to the patient and the moment in time when patient test response to the stimuli is detected if the patient test response occurs before a maximum test response time expires, otherwise calculating the timing to be the maximum test response time,
determining the correctness of the patient test response by comparing the patient test response to an expected test response,
recording the patient test response as a missed test response if the patient test response time is calculated as the maximum test response time, otherwise calculating additional patient test response data associated the patient test response, and
recording a correct test response and a correct test response timing if the expected test response occurred, otherwise recording an incorrect test response and an incorrect test response timing.

18. The method of claim 14, wherein determining, by the electronic processor, based on comparison of plurality of patient test-response records and a plurality of expected test-response records, a diagnosis of brain injury includes:
determining a number of correct, incorrect, premature, and missed test responses from the plurality of patient test responses,
calculating at least one statistical result from a plurality of patient test responses from the plurality of patient test-response records,
aggregating the plurality of patient test responses and the at least one statistical result from the plurality of patient test response timings,
comparing the plurality of patient test responses and the at least one statistical result from the plurality of patient test response timings to recorded responses from a pre-injury test of the patient if previous test results are available, otherwise comparing the plurality of patient test responses and the at least one statistical result from the plurality of patient test response timings to expected test responses from similar patients,
comparing correctness of patient test responses and timing of patient test responses across the plurality of patient test responses to determine consistency of patient test responses, and
integrating, at least, the correctness of patient test responses, the at least one statistical result, and the patient test results timings into patient test results to determine a diagnosis of brain injury.

19. The method of claim 18, further comprises transmitting within a 5-10 minute time period a set of 5-10 tests determining, by an electronic processor, the probability a brain injury has occurred based on patient test responses, and outputting to an output device a recommendation as to the need for additional testing to determine if a brain injury has occurred.
